(19) United States
(12) Patent Application Publication
(10) Pub. No.: US 2008/0300609 A1
(43) Pub. Date: Dec. 4, 2008

(54) ANASTOMOSIS DEVICE AND METHOD OF USING SAME

(76) Inventor: Bechara Tabet, Akron, OH (US)

Correspondence Address: FAY SHARPE LLP, 1100 SUPERIOR AVENUE, SEVENTH FLOOR, CLEVELAND, OH, 44114, US

(21) Appl. No.: 11/757,717

(22) Filed: Jun. 4, 2007

(51) Int. Cl. A61B 17/10

(52) U.S. Cl. 606/139

(57) ABSTRACT

An anastomosis device for reestablishing continuity to an associated human luminal structure at an associated damaged tissue region is provided. The device includes a first insert for supporting a first portion of the associated damaged tissue region. The first insert includes a generally cylindrical body, a tip, a base, and a bore extending through the body from the base to the tip. A second insert is provided for supporting a second portion of the associated damaged tissue region. The second insert includes a generally cylindrical body, a tip, a base, and a bore extending through the body from the base to the tip. An electromagnet is disposed in the tip of one of the first or the second insert for generating a magnetic field. A magnetically responsive member is disposed in the other of the first or the second insert. The member is attracted towards the electromagnet when the electromagnet is powered to an on state. Wherein the tips of the first and second inserts are adapted to self-align when electromagnetically engaged and hold together the first and second portions of the associated damaged tissue region to promote an anastomosis therebetween. A method is also provided.

### BACKGROUND

The present disclosure relates in various exemplary embodiments, to a method and device for repairing perforated or transected vessels or other luminal structures. The embodiments find particular application in conjunction with devices for performing anastomosis of the ureter and or urethra. However, it is to be appreciated that the present exemplary embodiments are also amenable to other like applications.

In the past, a number of techniques have been developed to rejoin transected or damaged vessels in the body. Generally, an incision is made in the body to reveal the damaged vessel. Once the damaged region is identified the ends of the vessel can be spatulated or cut diagonally. A stent is then optionally inserted into the damaged ends of the vessel. Naturally, the size and length of the stent depends on the vessel to be repaired. The ends are then stitched about the stent. Absorbable sutures may or may not be used. Thereafter, the primary incision is sutured and in the ensuing weeks the patient is allowed to recover. At some point after the procedure, X-ray imaging may be performed to determine if any leaks exists at the site of the anastomosis.

With respect to injuries of the ureter or urethra, the procedure used to repair the damaged regions varies somewhat with the precise location along the ureter or urethra (i.e. proximal, medial, or distal). However, in cases where anastomosis of the damaged ureter or urethra is the preferred, the procedure is similar to that described above.

Notwithstanding the relatively straightforward procedure of the prior art, several disadvantages do exist. For example, a large external incision is usually necessary which leads to longer recovery periods and increased discomfort for the patient. Also, placement of the stent and suturing of the ends of smaller more delicate vessels can be extremely tedious and time consuming. This results in longer surgeries, increased risks and recovery time for the patient, and increased risk of restenosis of the vessel.

Thus, the present disclosure is directed to overcoming one or more of the aforementioned problems and others.

### SUMMARY

According to one aspect of the present disclosure, an anastomosis device for reestablishing continuity to an associated human luminal structure at an associated damaged tissue region is provided. The device includes a first insert for supporting a first portion of the associated damaged tissue region. The first insert includes a generally cylindrical body, a tip, a base, and a bore extending through the body from the base to the tip. A second insert is provided for supporting a second portion of the associated damaged tissue region. The second insert includes a generally cylindrical body, a tip, a base, and a bore extending through the body from the base to the tip. An electromagnet is disposed in the tip of one of the first or the second insert for generating a magnetic field. A magnetically responsive member is disposed in the other of the first or the second insert. The member is attracted towards the electromagnet when the electromagnet is powered to an on state. Wherein the tips of the first and second inserts are adapted to self-align when electromagnetically engaged and hold together the first and second portions of the associated damaged tissue region to promote an anastomosis therebetween.

According to another aspect of the present disclosure, a method for performing an anastomosis of an associated human luminal structure at an associated damaged region is provided. The method includes the steps of providing a first insert including a body, a convex tip, a base and a bore extending through the body from the base to the tip. Providing a second insert including a body, a concave tip, a base and a bore extending through the body from the base to the tip. Providing an electromagnet in the tip of one of the first insert or the second insert capable of generating an electromagnetic field. Introducing the first insert into a first portion of the associated luminal structure. The first insert being arranged such that the convex tip portion is oriented in a direction towards the associated damaged region when the first insert is in a final position. Introducing the second insert into a second portion of the associated luminal structure. The second insert being arranged such that the concave tip portion is oriented towards the associated perforated site when the second insert is in a final position. Advancing the first insert until the tip of the first insert is near to the associated damaged region. Advancing the second insert until the tip of the second insert is near to the associated damaged region. Activating the electromagnet provided in the tip of one of the first insert or the second insert and generating the electromagnetic field. The electromagnet attracting a magnetically responsive member in the tip of the other one of the first insert or second insert. Aligning the first and second portions of the associated luminal structure at the associated damaged region by the convex tip of the one insert engaging the concave tip of the other insert and forming an anastomosis. Maintaining the first and second inserts in electromagnetic engagement while passing a guide wire through the respective bores of the first and second inserts. Deactivating the electromagnet. Withdrawing the first and second inserts. Inserting a catheter or stent over the guide wire and allowing the anastomosis to heal.

According to yet another aspect of the present disclosure, a ureter or urethral anastomosis device for reconstituting an associated damaged human ureter or urethra is provided. The device including a first insert for supporting a first portion of the associated damaged ureter or urethra. The first insert includes a generally cylindrical body, a tip, a base, and a bore extending through the body from the base to the tip. A second insert is provided for supporting a second portion of the associated damaged ureter or urethra. The second insert includes a generally cylindrical body, a tip, a base, and a bore extending through the body from the base to the tip. An electromagnet is disposed in the tip of one of the first insert or the second insert for generating an electromagnetic field. A magnetically responsive member is disposed in the tip of one of the other of the first insert or second insert. The responsive member being attracted towards the electromagnet when the electromagnet is powered to an on state. Wherein the tip of the first and second inserts are electromagnetically engaged to realign the first and second portions of the ureter to promote an anastomosis therebetween.

According to still another aspect of the present disclosure, an obturator for removing a stricture in an associated luminal structure is provided. The obturator includes a first body including a tip and a base, the tip includes an orifice and the base includes a port for receiving a first fluid. The orifice and the port are in fluid communication. A first inflatable balloon is disposed at the tip of the first body. A second body includes a tip and a base. The tip includes an orifice and the base including a port for receiving a second fluid, the orifice and the port being in fluid communication. A second inflatable balloon is disposed at the tip of the second body. Wherein, the second balloon is disposed below the first balloon and the second body is slidably engaged with the first body.

According to yet another aspect of the present disclosure, a method for removing a stricture from an associated human luminal structure is provided. The method includes the steps of providing an insert including an insert body, a tip, a base and a bore extending through the insert body from the base to the tip. Providing a dual balloon obturator including a first body having a first inflatable balloon and a second body having a second inflatable balloon. The second balloon being disposed below the first balloon and the first body being slidably engaged within the second body. Inserting the obturator into the bore of the insert with the first and second balloons in a deflated state. Inflating the second balloon. Introducing the insert and the obturator into a proximal portion of the associated luminal structure. Advancing the insert and the obturator until the tip of the insert is proximal to the associated stricture. Deflating the second balloon. Retracting the second body until the second balloon is disposed within the bore of the insert. Advancing the first balloon through the associated stricture. Inflating the first balloon. Retracting the first body and the first balloon and urging the associated stricture into a concave recess in the tip of the insert. Activating a suturing device within the insert and joining the proximal portion of the associated luminal structure adjacent the associated stricture to a distal portion of the associated luminal structure adjacent the associated stricture. Separating the associated stricture from the adjacent proximal and the distal portions of the associated luminal structure. Withdrawing the insert and obturator from the associated luminal structure.

Other benefits and advantages of the embodiments of the present disclosure will become apparent to those of average skill in the art upon a reading of the following detailed specification.

### BRIEF DESCRIPTION OF THE DRAWINGS

The method and device of the present disclosure may take form in certain structures and components, several non-limiting embodiments of which will be described in detail in this specification and illustrated in the accompanying drawings. In the drawings:

FIG. 1 is a perspective view of a first embodiment of an anastomosis device for reconstituting damaged luminal structures (e.g. vessels) including a first insert and a second insert, according to the present disclosure.

FIG. 2 is a cross sectional view of the anastomosis device of FIG. 1, illustrating a through bore and an electromagnet.

FIG. 3A is a partial cross sectional diagram of a male urinary system illustrating a bladder, a prostate, and a damaged urethra (e.g., ruptured, structured, or transected urethra) having the anastomosis device of FIG. 1 inserted therein.

FIG. 3B is a partial diagram of a male or female urinary system including a kidney, a damaged ureter, and a bladder is shown having the anastomosis device of FIG. 1 inserted within the damaged ureter at a proximal location with respect to the kidney.

FIG. 3C is a partial diagram of a male or female urinary system of FIG. 3B having the anastomosis device of FIG. 1 inserted within a damaged ureter at a distal location with respect to the kidney.

FIG. 4 is a side view of the anastomosis device of FIG. 1 illustrating the first and second inserts electromagnetically engaged.

FIG. 5 is a side view of a single balloon obturator, illustrated in an inflated state, for introducing the second insert into a luminal structure atraumatically.

FIG. 6 is a side view of the obturator of FIG. 5 illustrated in a deflated state.

FIG. 7 is a perspective view of the second insert of the anastomosis device configured with the single balloon obturator of FIG. 5.

FIG. 8 is a cross sectional view of the second insert as shown in FIG. 7.

FIG. 9 is a perspective view of a second embodiment of an anastomosis device, according to the present disclosure, illustrating a second insert having an integrated suturing assembly.

FIG. 10 is a cross sectional view of the anastomosis device of FIG. 9, illustrating a suture compartment, a suture trigger, and a suture driver of the suturing assembly.

FIG. 11 is a side view of a dual balloon obturator, for use with the second insert of FIG. 1, illustrating a first and a second balloon in an inflated state.

FIG. 12 is a side view of the obturator of FIG. 11 illustrating the first and second balloon in a deflated state.

FIG. 13A is a perspective view of the second insert of FIG. 9 and the dual balloon obturator of FIG. 11 being introduced into a strictured vessel during a stricture removal procedure.

FIG. 13B is a perspective view of the second insert and dual balloon obturator of FIG. 13A, illustrating the second balloon retracted within the second insert and the first balloon passing from a proximal side to a distal side of the stricture in the vessel.

FIG. 13C is a perspective view of the second insert and the dual balloon obturator of FIG. 13B, illustrating the first balloon inflated on the distal side of the stricture.

FIG. 13D is a perspective view of the second insert and the dual balloon obturator of FIG. 13C, illustrating the first balloon being withdrawn toward the second insert and compressing the stricture therebetween while an electromagnet of the second insert is activated.

FIG. 13E is a perspective view of the second insert and the dual balloon obturator of FIG. 13D, illustrating the first balloon fully seated against a tip end of the second insert while the stricture is removed and the proximal and distal ends of the vessel are reattached via a stapling unit.

### DETAILED DESCRIPTION

The present disclosure relates to a method and device for repairing damaged or transected luminal structures of the body, such as a ureter or urethra. The device includes a first insert for supporting a first portion of an associated damaged tissue portion. The first insert includes a generally cylindrical body, a tip, a base, and a bore extending through the body from the base to the tip. A second insert is provided for supporting a second portion of the associated damaged tissue region. The second insert includes a generally cylindrical body, a tip, a base, and a bore extending through the body from the base to the tip. An electromagnet is disposed in the tip of the second insert for generating a magnetic field. A magnetically responsive member disposed in the tip of the first insert. The member becomes attracted towards the electromagnet when the electromagnet is powered to an on state. Wherein, the tips of the first and second inserts are electromagnetically engaged to realign and hold together the first and second portions of the damaged tissue region to promote anastomosis therebetween.

It should be noted that the damaged tissue region may include a damaged vessel, a wall surface of another organ (e.g. kidney, bladder, bowel, etc) or any other hollow or luminal structure. The damaged tissue region generally includes the area or region of tissue where a rupture, stricture, transection, perforation, genetic deformity, or other damage has occurred. In addition, the present disclosure is not limited to repairing or reconstituting damaged luminal structures but could also be used in instances when a new connection is desired where no connection existed previously. For example, as in the case of a colon repair after a colectomy.

The exemplary embodiments of this disclosure are more particularly described below with reference to the drawings. Although specific terms are used in the following description for clarity, these terms are intended to refer only to the particular structure of the various embodiments selected for illustration in the drawings and not to define or limit the scope of the disclosure. The same reference numerals are used to identify the same structure in different Figures unless specified otherwise. The structures in the Figures are not drawn according to their relative proportions and the drawings should not be interpreted as limiting the disclosure in size or location. In addition, as will be appreciated by those skilled in the art, the present disclosure is applicable to both female and male anatomies and should not be limited to one or the other based on the examples presented herein.

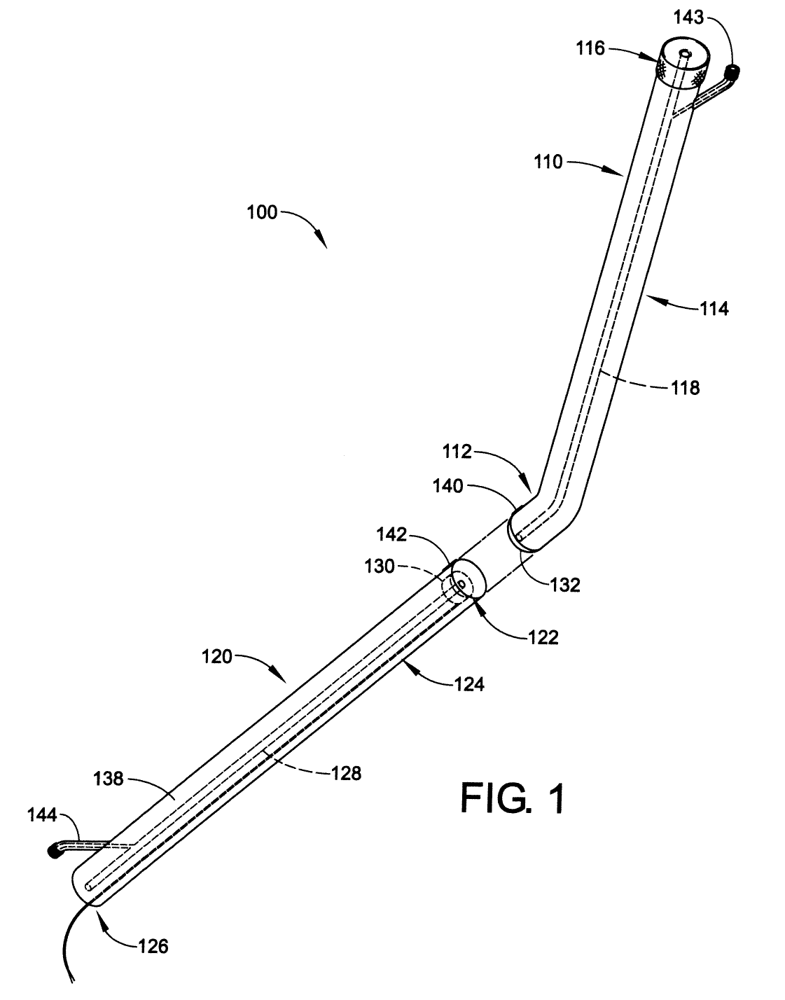

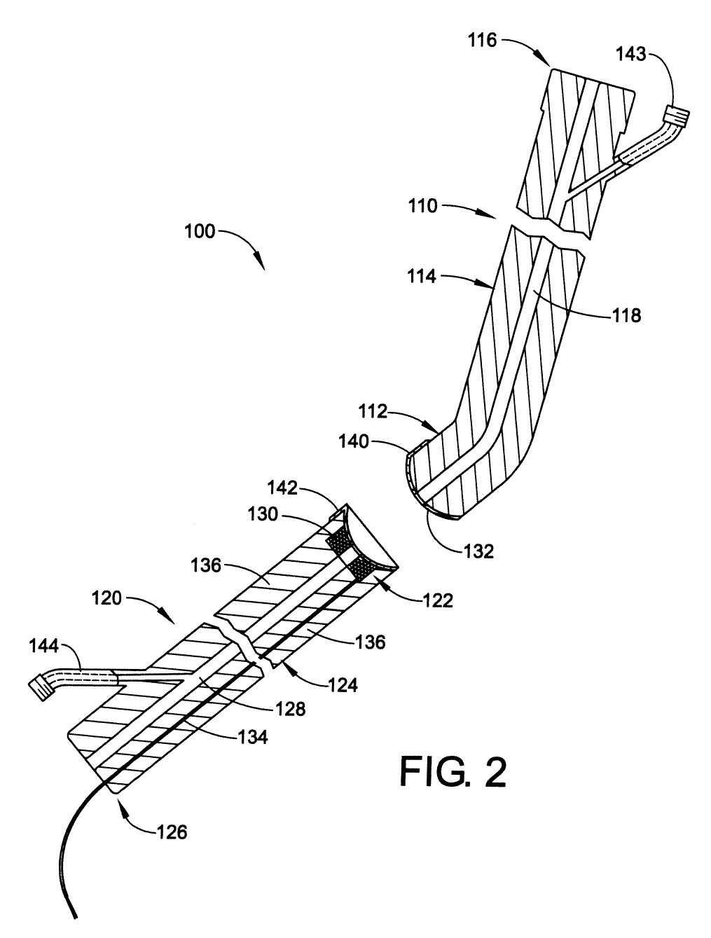

With reference to FIGS. 1 and 2, a first embodiment of an anastomosis device 100 is shown. Generally, the device 100 includes a first insert 110 having a tip 112, a generally cylindrical body 114 and a base portion 116. The first insert 110 also includes a bore 118 which extends from the base portion 116 to the tip 112. The anastomosis device 100 further includes a second insert 120 which also includes a tip portion 122, a generally cylindrical body portion 124, a base portion 126 and a bore 128 extending from the base portion 126 towards the tip portion 122. In addition, the second insert 120 includes an electromagnet 130 disposed in the tip portion 122. Similarly, a magnetically responsive member 132 is disposed in or along a tip portion 112 of the first insert 110. The electromagnet 130 receives electrical power via a wire 134 which may be imbedded within a wall 136 defined between the bore 128 and an outer surface 138 of the first insert. Furthermore, the first and second inserts 110, 120 include an indexing tab 140, 142 (FIGS. 1-4) disposed about the tip portions 112, 122, respectively. The indexing tabs can be attached to an outer surface of the insert or can be embedded within the inserts. Furthermore, the indexing tabs may be made from any material that is visible under fluoroscopy such that an operator or surgeon can easily determine the rotational or angular orientation of the inserts after they have been introduced into the patient's body. In addition, a first insert injection port 143 is provided at the base 116 of the first insert 100 and a second insert injection port 144 is provided at the base 118 of the second insert 120. The respective injection ports 143,144 may be adapted to receive a syringe or other device capable of dispensing a fluid (e.g. a contrast dye) for facilitating the navigation, placement, or alignment of the inserts or for dispensing a medication within the tissue or region to be repaired.

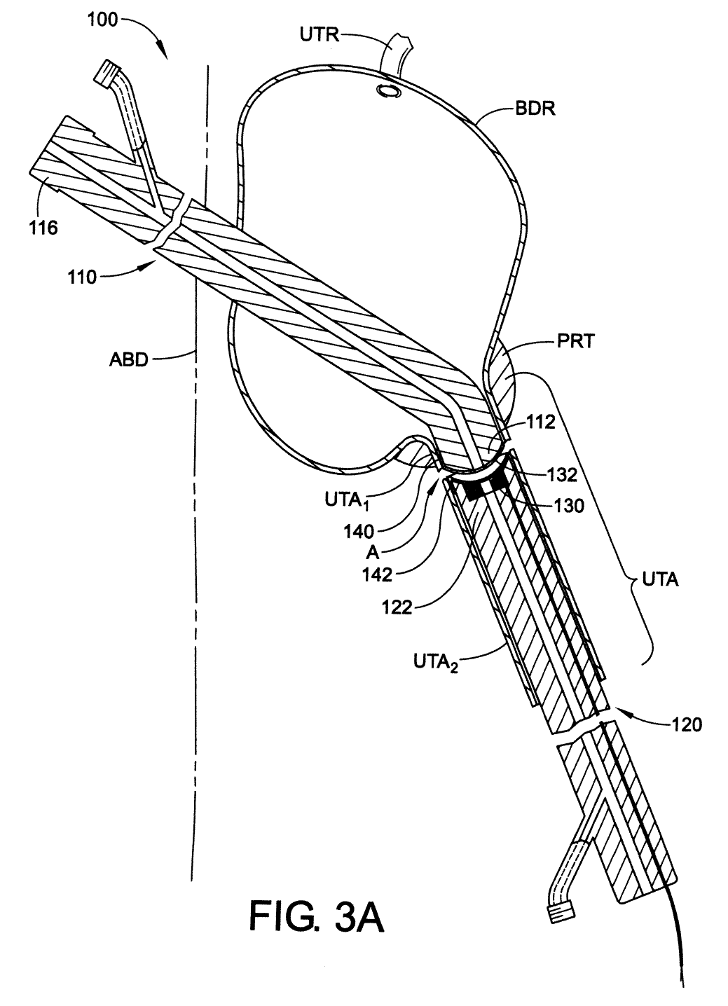

Now with reference to FIG. 3A, and by way of example only, the anastomosis device 100 is illustrated as being implemented in a male urinary system. The partial cross section of the male urinary system includes a ureter UTR, a bladder BDR, a prostate PRT, and a urethra UTA. Specifically, the urethra UTA of the instant example is severed adjacent the prostate PRT proximal to the bladder BDR about a region A. In placing the inserts a set of guide wires (not illustrated), one for each insert, is first introduced from an access point (back, abdomen, urethra, etc.) and fed through the appropriate vessels, organs, or structures until each is in the target or final position along the damaged region. Once the guide wires are in position, each of the first and second inserts are slid over the respective guide wires until each is in the correct position. In the instant example, a guide wire (not shown) is first introduced percutaneously under fluoroscopy through an abdominal wall ABD, into the bladder BDR and into the urethra UTA. The first insert 110 is then slid over the guide wire through the abdominal wall ABD and into the bladder BDR. In negotiating the various curvilinear portions of the vessel or tissue to be repaired, it may be beneficial for one or both tips to have a slightly angled presentation with respect to a longitudinal axis of the body of the insert. Here, only the tip portion 112 of the first insert 110 is angled. Because the tip 112 is angled, the first insert 110 is more easily navigated into a proximal or upper portion UTA₁ of the urethra UTA that is proximal to the bladder BDR. Furthermore, the base 116 of the first insert 110 can be textured or enlarged, the operator or surgeon can easily rotate, pull, or push as necessary to negotiate the tip 112 of the first insert 110 into the upper portion UTA₁ of the urethra UTA.

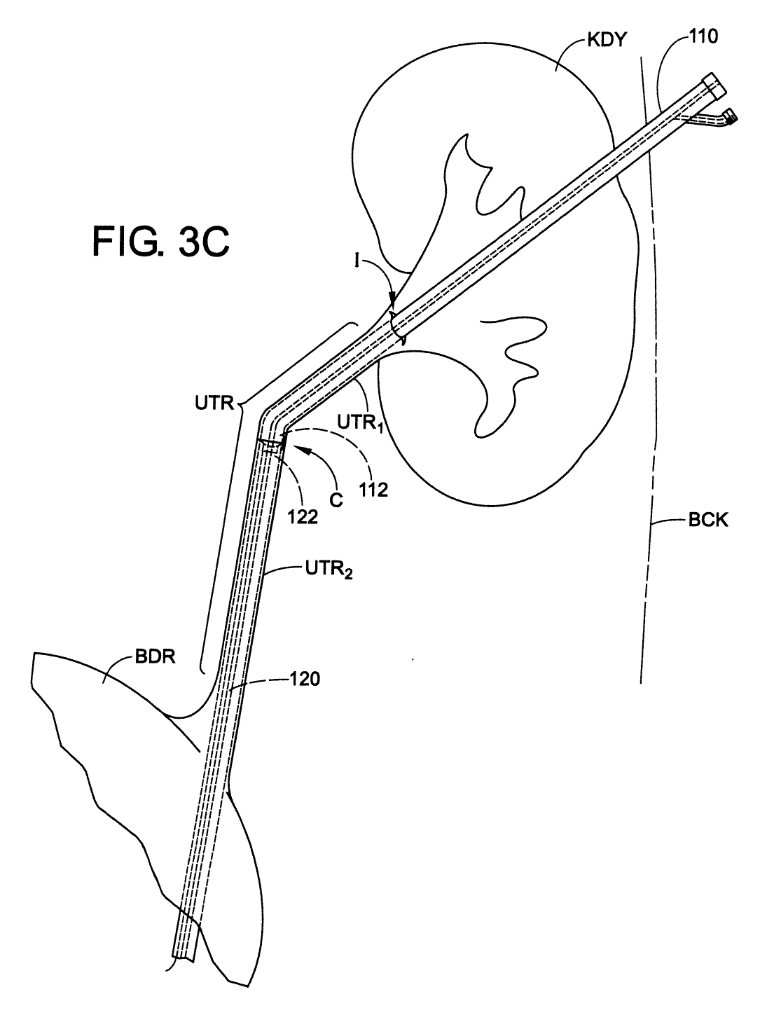

With continued reference to FIG. 3A, next the second insert 120 can be introduced through the urethral meatus or distal portion UTA₂ of the urethra UTA and advanced towards the severed region A. If a complete urethral transection is to be treated, a guide wire will be introduced into the bore of one insert (from the base portion) and passed in the bore of the other insert (through the tip end) after the inserts have been magnetically engaged. To prevent trauma or other tissue damage to the urethra UTA while introducing the second insert 120 (due to the relatively sharp edge of the concave tip 122) a balloon tipped obturator 145 (FIG. 5) may be used in conjunction with the second insert 120. If used, the obturator would be placed into the second insert 120 while in a deflated state and then inflated prior to introduction of the insert 120 into the urethra UTA. Once the second insert 120 has been introduced into the urethra UTA and is close to its final position or severed region A, the obturator is then deflated and withdrawn. A more detailed discussion of the obturator is provided below with reference to FIGS. 5-8. In addition to the use of one or more guide wires, the final positioning and rotational orientation of the inserts 110,120 can be easily accomplished using the indexing tabs 140,142. Specifically, the surgeon can make minor alignment or other adjustments by rotating the inserts 110,120 until the indexing tabs 140,142 become visibly aligned under fluoroscopy.

Once the tip portions 112, 122 are relatively close, the electromagnet 130 is then energized which causes the magnetically responsive member 132 to be drawn towards the electromagnet 130. Simultaneously, the respective tip portions 112, 122 draw the proximal portion UTA₁ of the urethra UTA into contact with the distal portion UTA₂. Once this occurs, a relatively tight seal is formed therebetween. The nearly gapless positive engagement of the inserts thus increases the probability of a successful anastomosis or alignment of the damaged end portions UTR₁, UTR₂. In addition, the convex shape of the first tip portion 112 and the concave shape of the second tip portion 122 aids in self-aligning the tip portions 112,122 with respect to a longitudinal axis of the urethra UTA. Once the proximal and distal portions UTA₁, UTA₂ of the urethra UTA are set, the inserts 110, 120 maybe removed leaving only a single guide wire in place. A council tip Foley catheter may then be introduced over the guide wire to assure continuity of the urinary tract. Alternately and in case of a stricture, a suturing assembly (as will be described in greater detail below) may be used to staple or otherwise physically reattach the severed ends of the urethra prior to removal of the inserts.

With reference to FIG. 3B, a partial diagrammatical sketch of a male or female urinary system is shown, including a kidney KDY, a bladder BDR and a ureter UTR. The ureter UTR includes a damaged region B. As in the previous example of FIG. 3A, the first and second inserts 110, 120 can be received individually into the luminal structure to be repaired (or the ureter UTR) with [or without] the use of one or more guide wires. In the instant example, the first insert 110 is introduced via a back BCK of the patient and through an incision I in a proximal portion UTR₁ of the ureter UTR proximal to the kidney KDY. On the other hand, the second insert 120 is inserted via the urethra (not shown) through the bladder BDR and into a distal portion UTR₂ of the ureter UTR (with respect to the kidney). Eventually, the respective inserts are advanced towards the damaged region B. Once the tips 112,122 of the inserts 110,120 are brought in close proximity, the electromagnet within the second insert 120 is activated. Upon activation, the magnetically responsive member of the first insert 110 is drawn toward the second insert, thus bringing a segment of healthy tissue from each of the proximal UTR₁ and distal UTR₂ portions into contact with one another. At this point, staples may be used to attach the healthy ends of the proximal UTR, and distal UTR₂ portions. Once the ends are properly secured, the inserts 110, 120 are withdrawn and the patient begins recovery. Since almost the entire procedure is done internally or percutaneously, rather than external to the body (which would require a large incision site) the recovery period and discomfort to the patient are minimized. Lastly, it should be noted that invention of the present disclosure could be used for other types of luminal structure problems such as complete transections, relocations, strictures, ruptures, tears, perforations or genetic deformities.

With reference now to FIG. 3C, a male or female urinary system, similar to that described above and depicted in FIG. 3B, is shown. However, a damaged region C is depicted at a more distal location from the kidney KDY. Although, damage to the ureter UTR in the general area of the damaged region C can and does occur, it is not as common as the scenarios described above with respect to FIGS. 3A and 3B. Nevertheless, the instant procedure is nearly identical to that described above with reference to FIG. 3B. The first insert 110 can be introduced via the back BCK of the patient and through the incision I in the proximal portion UTR₁ of the ureter UTR proximal to the kidney KDY. The second insert 120 is then introduced via the bladder BDR and through the distal portion UTR₂ of the ureter UTR. The respective tips 112, 122 of the inserts 110,120 are navigated and eventually located proximally to the damaged region C of the ureter UTR. By electrifying the electromagnet 130 within the second insert 120, a magnetic field is generated which attracts the magnetically responsive member 132 disposed in the first insert 110. As such, the healthy portions of the luminal structure or ureter UTR are brought together and attached using the techniques described previously or any other available means.

It should be noted that the magnetically responsive member can be an embedded metal or powder, a permanent magnet, or any other material having ferro-magnetic type properties. Furthermore, the magnetically responsive member could comprise a secondary electromagnet. In this case, the secondary electromagnet could be configured to be manually activated before or after the activation of the first electromagnet. Alternately, the secondary electromagnet could be activated via a pair of electrical pressure contacts such that when the tip of the first insert contacts the tip of the second insert an electrical connection is made.

By way of example, the electromagnet is illustrated herein as being disposed within the second insert, whereas the magnetically responsive member is disposed within the first insert. However, it should be understood by those having skill in the art that the electromagnet and the magnetically responsive member could be swapped or rearranged in order to accomplish a similar effect. Naturally, the inserts must be close enough to one another such that the electromagnetic field will be adequately intense to draw the two inserts together.

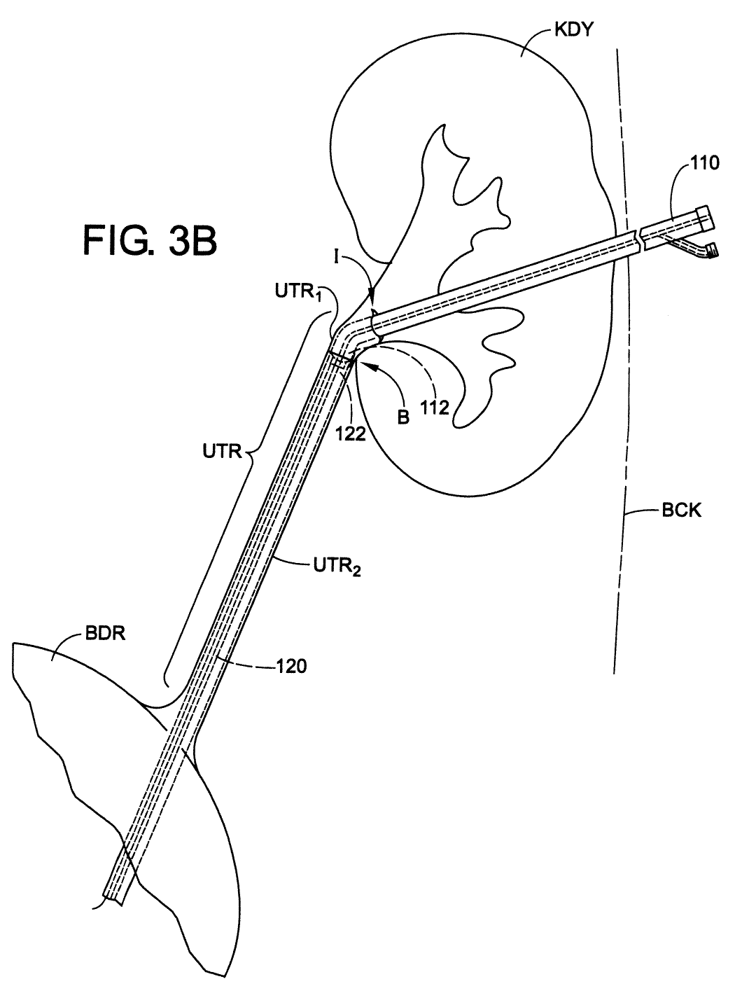

With reference to FIG. 4, an enlarged detail of the anastomosis device 100 of FIG. 1 is shown. As illustrated, the first insert 110 is shown electromagnetically engaged to the second insert 120 along the tip portions 112, 122. Furthermore, the indexing tabs 140,142 are also shown in alignment. It should be noted that the bores 118,128 of the first and second inserts 110,120 are aligned when the first and second inserts 110,120 are engaged. The bores 118,128 are primarily intended to be used with the guide wires for alignment and navigational purposes. In addition, when the bores are aligned they also permit a guide wire or fluid to communicate between the base portions 116, 126 of the first and second inserts 110, 120. Optionally, the bores 118,128 may also be used to allow fluid injection to occur into the damaged region or anastomosis site.

As shown in FIG. 5, a single balloon obturator 145 is shown in an inflated state. The obturator 145 includes an inflatable balloon tip 146 having a plurality of biasing ribs 148, a central tubular portion 150, and a base portion 152. The obturator 145 is designed to be used with the second insert 120 (FIGS. 1-4) to prevent damage to the interior wall of the luminal structure by the tip of the second insert. The base portion 152 may include a valve (not shown) allowing the balloon tip 146 to be inflated by a pressurized fluid supply and to be subsequently disconnected from the fluid supply without losing pressure inside the balloon tip 146. As such, the tubular portion 150 allows fluid communication between the base portion and the balloon tip. Because the tubular portion 150 is located within the bore 128 of the second insert, the base 152 and the balloon tip 146 serve to trap the inflated obturator 145 about the second insert. As an additional benefit, the ribs 148 could include magnetically responsive properties. If magnetically responsive ribs (in the obturator) are used and the obturator is used in combination with the insert, the electromagnet of the insert could be activated during introduction to maintain the balloon 146 in positive and secure contact with the tip of the insert.

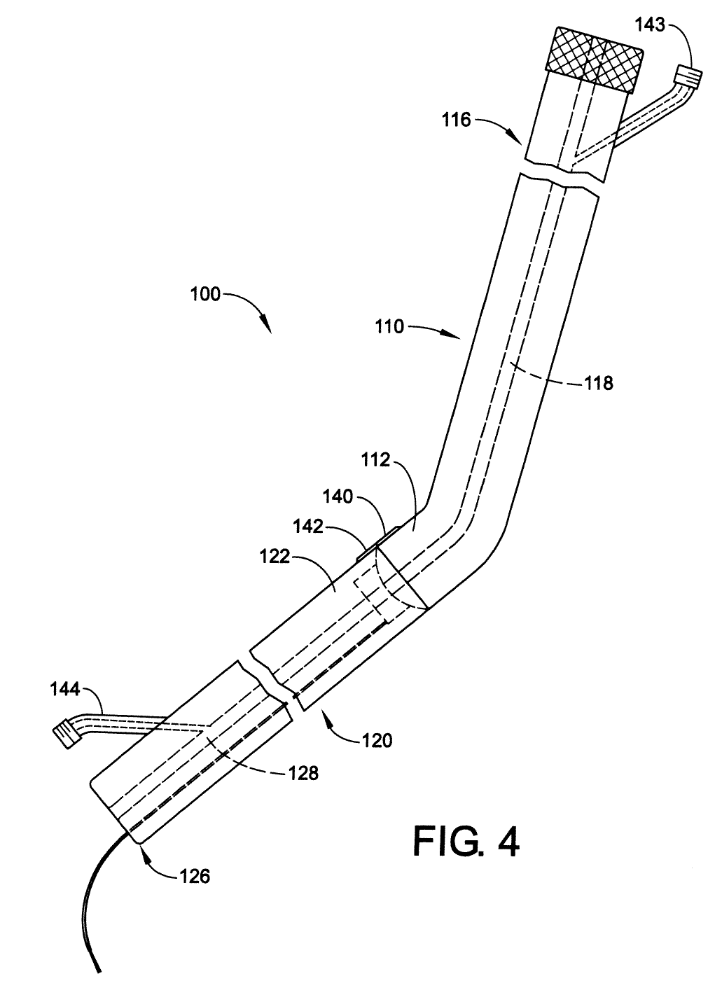

With respect to FIG. 6, the obturator 145 is shown in a deflated state. When the obturator 145 is in the deflated state, the balloon tip portion 146 may be easily inserted through the bore of the second insert. Similarly, when the second insert has been placed in its final position and just prior to electromagnetic engagement of the first and the second inserts, the valve 152 can be opened. The valve in the base 152 can be released using a Luer lock-tip syringe thus permitting the balloon tip 146 to deflate under the biasing force of the ribs 148. The obturator 145 can then be drawn outward and away from the inserts, thus permitting the concave tip portion of the second insert to engage the convex tip portion of the first insert.

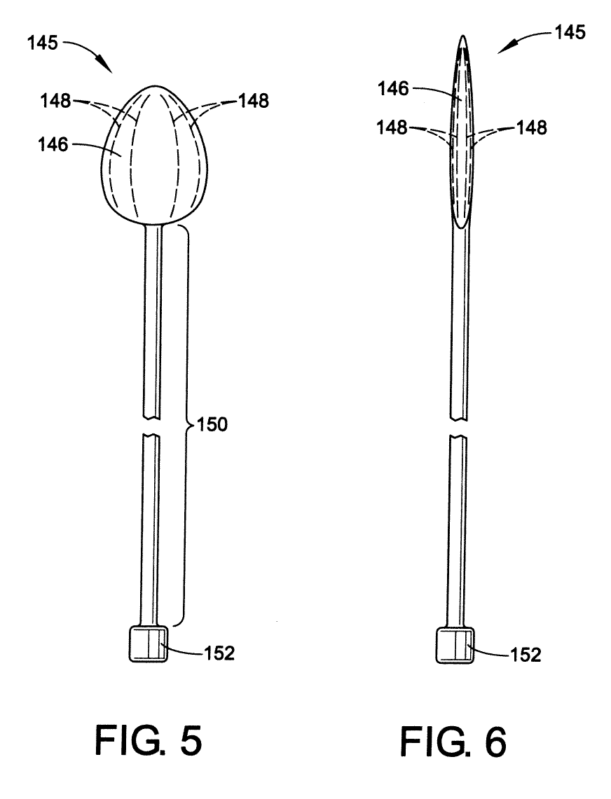

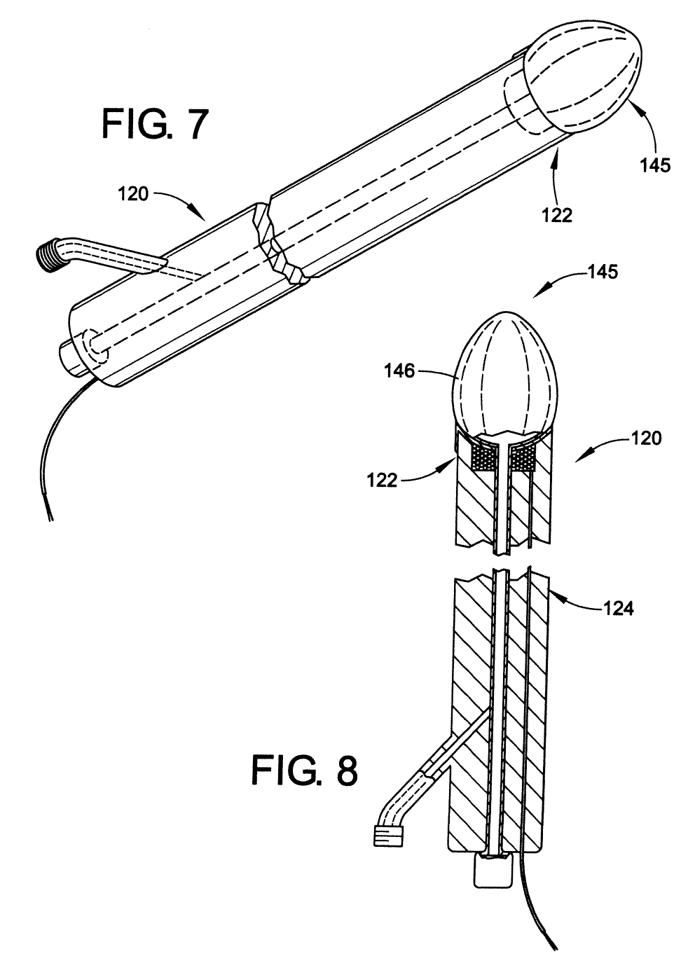

With reference to FIGS. 7 and 8, the second insert 120 is shown as having the obturator 145 installed therein. As mentioned previously, this configuration is generally preferred prior to introduction of the second insert 120 into the luminal structure to be repaired to avoid trauma to the luminal structure and or other related tissue structures. Because an outer circumferential portion of the concave tip 122 forms a relatively sharp edge, significant lacerations and or abrasions can occur within the luminal structure as the insert 120 is introduced and advanced towards the damaged region of the luminal structure. As shown in FIG. 8, by inserting the obturator 145 into the second insert 120 the balloon 146 of the obturator 145 inflates to form a generally spherical or egg-like shape. In addition, the maximum transverse diameter of the balloon 146 (when in the inflated state) can be as large as the diameter of the body 124 or tip 122 portions. As such, a luminal structure or other tissue structure will not come into contact with the outer circumferential portion of the tip 122 during introduction and traumatic tissue damage can be avoided.

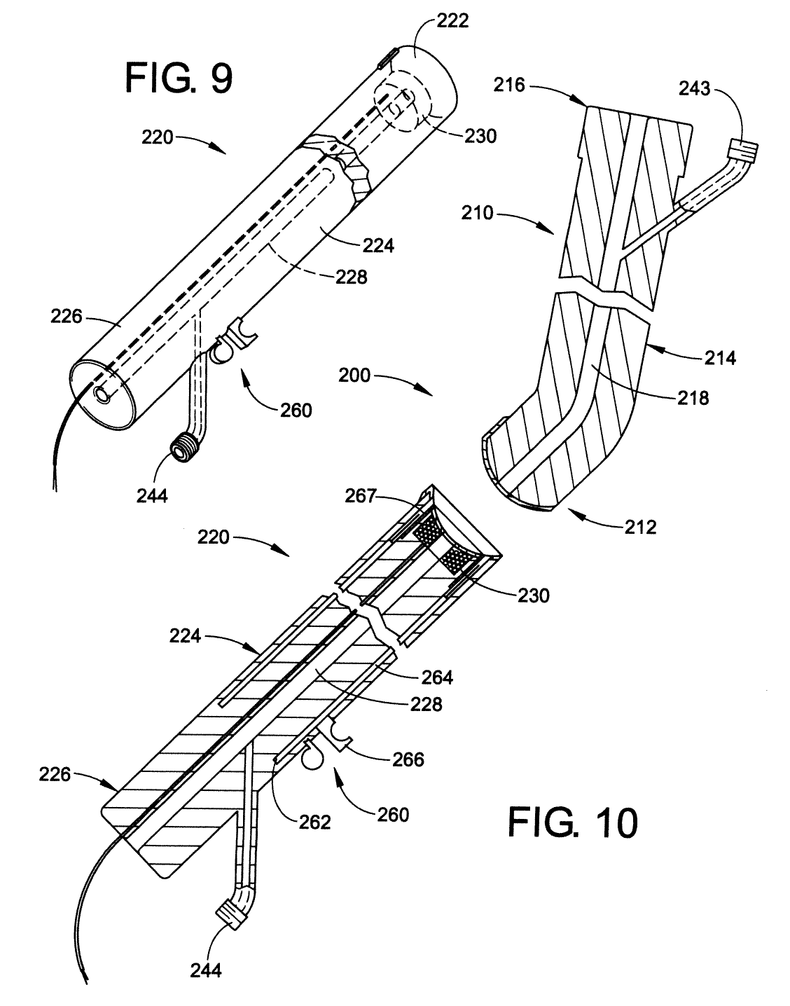

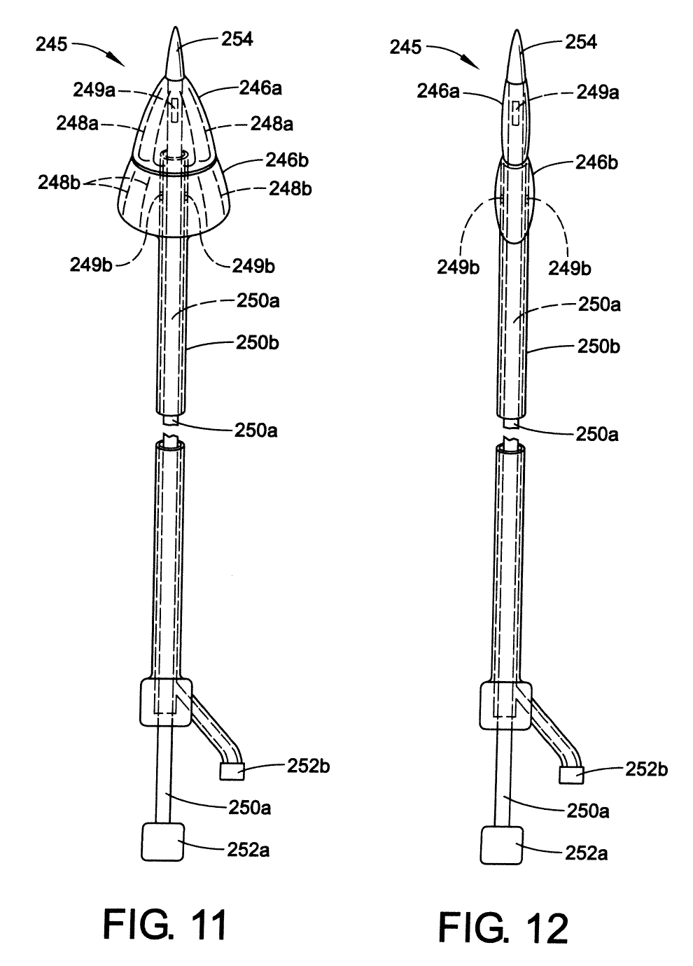

With reference to FIGS. 9-10, a second embodiment of an anastomosis device 200 is shown. The anastomosis device 200 includes a first insert 210 and a second insert 220 having similar features as that of the first embodiment. The inserts 210,220 include a tip portion 212,222, a body portion 214,224, a base portion 216,226, and a bore 218,228. In addition, the second insert 220 includes an electromagnet 230 for electromagnetically engaging a magnetically responsive member in the first insert 210. Furthermore, injection ports 243, 244 are provided for injecting fluids near the tip portions 212,222 into the body of the patient during a procedure. However, the primary difference with the anastomosis device 200 involves the use of a suturing assembly 260 for suturing together the healthy end portions of the luminal structure, vessel, or other tissue to be repaired.

With particular reference to FIG. 10, a cross section of the anastomosis device 200 is shown with the suturing assembly 260. The suturing assembly 260 includes a suture compartment 262 for receiving an appropriate suture. It should be noted that the terms “suture” or “suturing”, as defined herein, are not to be construed as being limited to conventional materials and techniques for closing a surgical site (e.g. stitches or staples) but may also include any material and or technique used to secure one tissue structure to another. As illustrated in FIG. 10, one or more staples may be loaded into the suture compartment 262. A suture driving element 264 is disposed adjacent the suture compartment 262 for driving the one or more staples about the anastomosis or the damaged end portions of the luminal structure. The suture driver 264 can be configured to insert a suture upon activation of the suture trigger 266 while the first and second inserts (and the damaged end portions) are held in place via the electromagnet 230. When staples are used, the tip 212 of the first insert 210 may serve as an anvil or a reaction surface for the staples to react against as they are being ejected. As the sutures (or staples) are delivered, the end portions of the tissue structure are securely attached. Once the end portions are properly attached, a resection of the excess or damaged portions of the anastomosis can be performed by extending a circular cutter 267 that is slidably engaged or otherwise disposed in the tip portion 222 of the second insert 220.

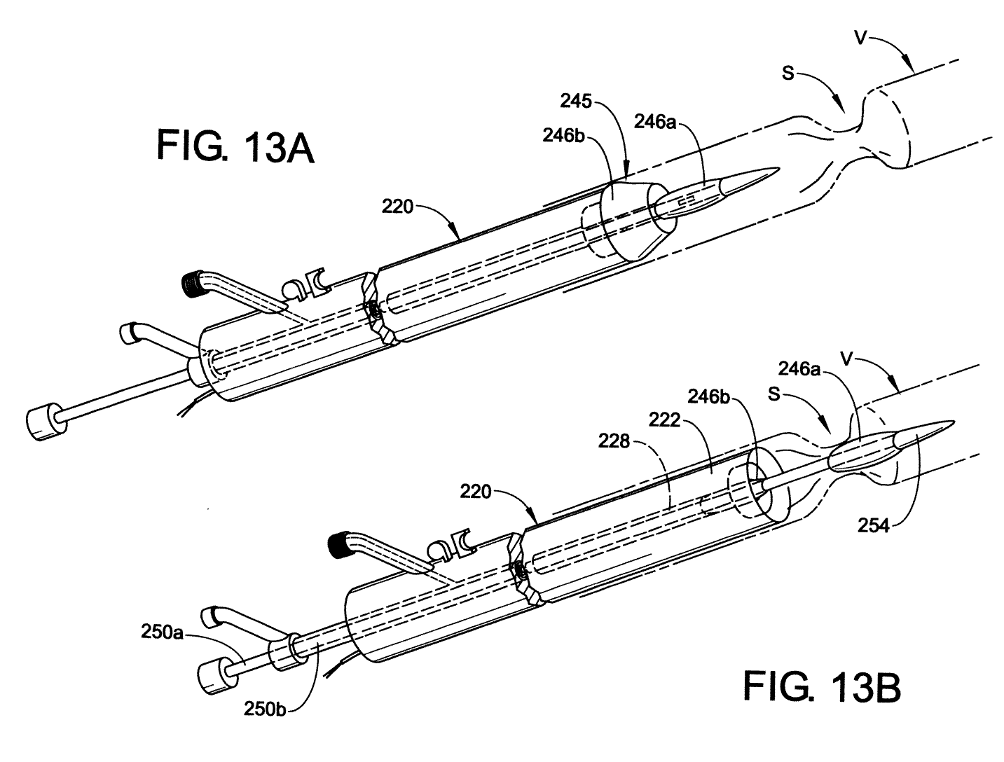

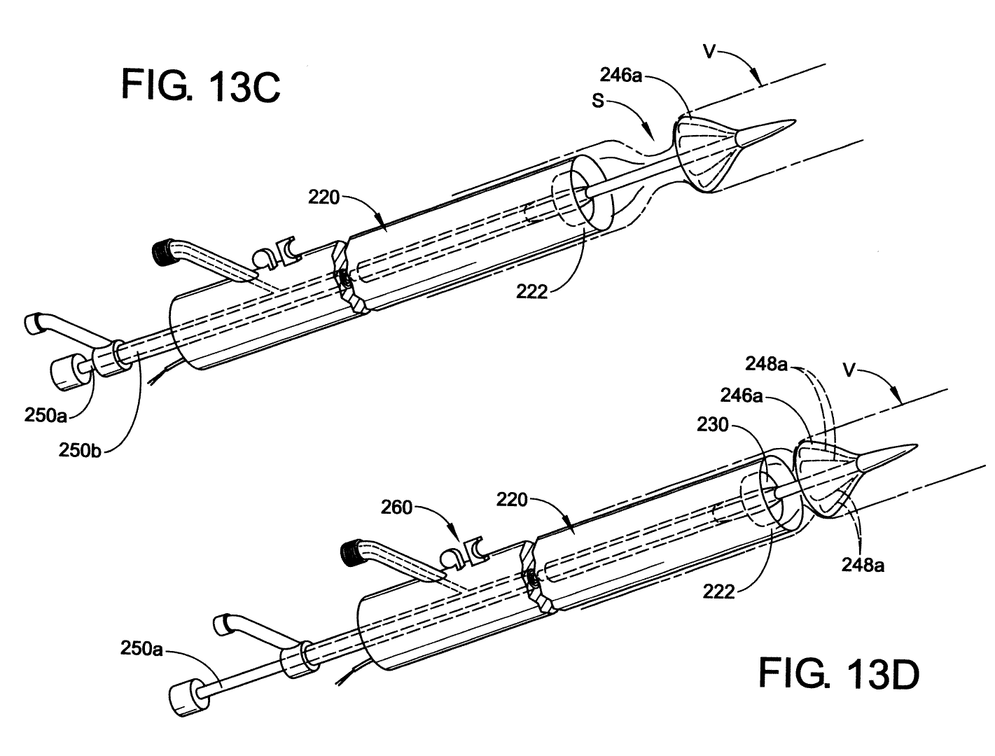

With reference now to FIGS. 11 and 12, a dual balloon obturator 245 for use with the second insert of the second embodiment of the anastomosis device is shown. The dual balloon obturator 245 includes a first and a second balloon 246a, 246b having a set of stiffening ribs 248a, 248b, respectively. One or more of the stiffening ribs may be magnetically responsive. Each of the first and second balloons 246a, 246b includes one or more orifices 249a, 249b for inflating and/or deflating the respective balloons. The first and second balloons 246a, 246b are attached at the tip end 254 of the corresponding bodies 250a, 250b. In addition, the inner or first body 250a is slidably engaged within the outer or second body 250b.

Furthermore, each of the respective first and second bodies 250a, 250b, include a fluid communication channel such that the first and second balloons 246a, 246b may be independently inflated and/or deflated via the orifices 249a, 249b. The orifices 249a, 249b are in respective communication with the first and second valve bodies 252a, 252b. As with the single balloon obturator 145 of FIG. 5, the dual balloon obturator 245 can be used with the second insert of the anastomosis device for preventing atraumatic introduction into and through the luminal structure to be repaired. However, the dual balloon obturator 245 is particularly adapted to perform a stricture removal procedure. As will be discussed in greater detail below, the stricture removal procedure can be performed with only a single insert, rather than two inserts as shown in FIGS. 3A-3C.

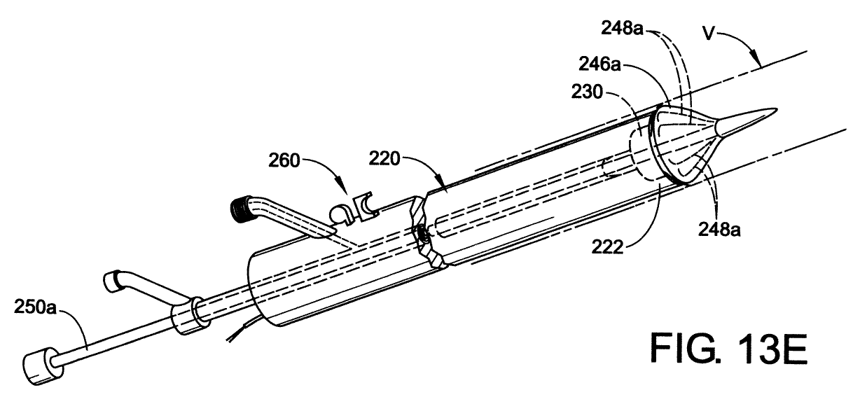

With reference to FIG. 13A, the second insert 220 is shown partially introduced into a malformed or damaged luminal structure such as a vessel V having the dual balloon obturator 245 attached thereto. In particular, and by way of example only, the vessel V includes a stricture S. Initially, the insert 220 and dual balloon obturator 245 are inserted with the first balloon 246a deflated and the second balloon 246b inflated. Introducing the obturator in this configuration has the same effect as using the single balloon obturator described previously. That is, it reduces vessel trauma during introduction of the second insert.

With reference to FIG. 13B, once the insert 220 is near to the stricture S, the second balloon 246b is deflated and the second body 250b is withdrawn such that the second balloon 246b recedes into the bore 228 within the tip portion 222. After the second balloon 246b is withdrawn into the bore 228, the insert 220 can be advanced even closer towards the stricture S within the vessel V. Eventually, the tip end 254, the first balloon 246a and the first body 250a are advanced completely through the stricture S. To facilitate this, the tip end 254 may come to a point or be conically shaped such that the first balloon 246a can be biased or urged through the stricture S without significant resistance. In addition, a previously placed guide wire can be used to guide the loaded insert.

With reference to FIG. 13C, once the first balloon 246a has cleared the strictured portion S, it may be inflated so as to entirely consume the normal diameter of the vessel V. The body 250a can then be retracted, while not disturbing the second body 250b, causing the distal portion of the vessel V to be drawn towards the tip end 222 of the second insert 220.

With reference to FIGS. 13D and 13E, once the tip portion 222 of the second insert 220 is proximal to the inflated first balloon 246a, the electromagnet 230 may be activated. Since the stiffening ribs 248a in the first balloon 246a are magnetically responsive, they are attracted to the electromagnet 230 when the electromagnet is energized. As such, the first balloon 246a is drawn completely against the tip portion 222 of the second insert 220. Simultaneously, the first body 250a is further retracted to compress the strictured portion even more using first balloon 246a. Accordingly, the strictured portion collapses into the concave tip portion 222 of the second insert 220. At this point, the proximal and distal portions of the vessel V can be stitched, stapled, or otherwise reattached using the suturing assembly 260. As such, the vessel V is securely reattached about the circumference of the tip end 222 of the second insert 220. In addition, as the suturing assembly reattaches the two portions of the vessel V, the strictured portion is partially cut due to the perforating nature of the individual sutures or staples. Then, the circular cutter that is disposed at the end of the insert and or integrated with the suturing assembly, can be used to completely cut away the strictured vessel portion. Afterwards, the stricture vessel portion can be easily detached and removed as the second insert 220 and the dual balloon obturator 245 are withdrawn from the vessel V.

The exemplary embodiment has been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

1. An anastomosis device for reestablishing continuity to an associated human luminal structure at an associated damaged tissue region, the device comprising:
a first insert for supporting a first portion of the associated damaged tissue region, the first insert including a generally cylindrical body, a tip, a base, and a bore extending through the body from the base to the tip;
a second insert for supporting a second portion of the associated damaged tissue region, the second insert including a generally cylindrical body, a tip, a base, and a bore extending through the body from the base to the tip;
an electromagnet disposed in the tip of one of the first or the second insert for generating a magnetic field;
a magnetically responsive member disposed in the other of the first or the second insert, the member being attracted towards the electromagnet when the electromagnet is powered to an on state; and
wherein the tips of the first and second inserts are adapted to self-align when electromagnetically engaged and hold together the first and second portions of the associated damaged tissue region to promote an anastomosis therebetween.

2. The device of claim 1, wherein the tip of one of the first or second insert is generally concave and the tip of the other of the first or second insert is generally convex.

3. The device of claim 1, wherein the tips of the first and second inserts further include an indexing tab.

4. The device of claim 1, wherein the tip of the first insert or the second insert includes an angular bend with respect to a longitudinal axis of the generally cylindrical body for introducing the respective first or second insert percutaneously into the respective associated damaged tissue region.

5. The device of claim 1, wherein the body further includes a suturing assembly.

6. The device of claim 1, wherein the bore of the first and second inserts are adapted to receive a guide wire for positioning the respective inserts and for maintaining the alignment of the respective first and second portions of the associated damaged tissue region.

7. The device of claim 1, wherein the bore of the first insert is at least partially aligned and in fluid communication with the bore of the second insert when the tips of the first and second inserts are electromagnetically engaged.

8. The device of claim 2, wherein the concave tip insert is adapted to receive a removable balloon tipped obturator for atraumatic insertion of the insert into the respective first or second portion of the associated damaged tissue region, the obturator having an inflatable balloon tip portion, a tubular central portion, and a bottom end portion, the bottom end portion including a valve for inflating or deflating the balloon tip portion.

9. The device of claim 8, wherein the balloon tip portion includes a magnetically responsive member.

10. The device of claim 1, wherein the body of one or both of the first and second insert include an injection port for introducing a fluid into the associated damaged tissue region.

11. A method for performing an anastomosis of an associated human luminal structure at an associated damaged region, the method comprising the steps of:
providing a first insert including a body, a convex tip, a base and a bore extending through the body from the base to the tip;
providing a second insert including a body, a concave tip, a base and a bore extending through the body from the base to the tip;
providing an electromagnet in the tip of one of the first insert or the second insert capable of generating an electromagnetic field;
introducing the first insert into a first portion of the associated luminal structure, the first insert being arranged such that the convex tip portion is oriented in a direction towards the associated damaged region when the first insert is in a final position;
introducing the second insert into a second portion of the associated luminal structure, the second insert being arranged such that the concave tip portion is oriented towards the associated perforated site when the second insert is in a final position;
advancing the first insert until the tip of the first insert is near to the associated damaged region;
advancing the second insert until the tip of the second insert is near to the associated damaged region;
activating the electromagnet provided in the tip of one of the first insert or the second insert and generating the electromagnetic field, the electromagnet attracting a magnetically responsive member in the tip of the other one of the first insert or second insert,
aligning the first and second portions of the associated luminal structure at the associated damaged region by the convex tip of the one insert engaging the concave tip of the other insert and forming an anastomosis;
maintaining the first and second inserts in electromagnetic engagement while passing a guide wire through the respective bores of the first and second inserts;
deactivating the electromagnet;
withdrawing the first and second inserts; and
inserting a catheter or stent over the guide wire and allowing the anastomosis to heal.

12. The method of claim 11, wherein the step of introducing the first and second inserts includes first inserting the guide wire from the first portion of the luminal structure to the second portion of the luminal structure and introducing the first and second inserts by sliding the first and second inserts over the respective ends of the guide wire.

13. The method of claim 11, wherein the step of introducing the second insert further includes inserting a balloon tip obturator into the second insert and inflating the balloon prior to introduction of the second insert into the second portion of the associated luminal structure.

14. The method of claim 13 wherein the step of advancing the second insert further includes deflating the obturator and removing the obturator from the second insert once the second insert is near to the associated damaged region.

15. The method of claim 11, further including the step of providing a suturing assembly in the second insert and applying one or more sutures to the associated damaged region while the first and second insert are electromagnetically engaged.

16. The method of claim 11, further including the step of withdrawing the guide wire after the catheter or stent has been inserted.

17. A ureter or urethral anastomosis device for reconstituting an associated damaged human ureter or urethra, the device comprising:
a first insert for supporting a first portion of the associated damaged ureter or urethra, the first insert including a generally cylindrical body, a tip, a base, and a bore extending through the body from the base to the tip;
a second insert for supporting a second portion of the associated damaged ureter or urethra, the second insert including a generally cylindrical body, a tip, a base, and a bore extending through the body from the base to the tip;
an electromagnet disposed in the tip of one of the first insert or the second insert for generating an electromagnetic field;
a magnetically responsive member disposed in the tip of one of the other of the first insert or second insert, the responsive member being attracted towards the electromagnet when the electromagnet is powered to an on state; and
wherein the tip of the first and second inserts are electromagnetically engaged to realign the first and second portions of the ureter to promote an anastomosis therebetween.

18. The anastomosis device of claim 17, wherein the tip of the first insert is generally convex and the tip of the second insert is generally concave.

19. The anastomosis device of claim 17, wherein the tips of the first and second inserts further include an indexing tab visible under fluoroscopy.

20. The anastomosis device of claim 17, wherein at least one of the tips of the first or second insert includes a bend with respect to a longitudinal axis of the respective body.

21. The anastomosis device of claim 17, wherein the body of the first or second insert further includes a suturing assembly.

22. The anastomosis device of claim 17, wherein the bore of the first insert is at least partially aligned and in communication with the bore of the second insert when the tips of the first and second inserts are electromagnetically engaged.

23. The anastomosis device of claim 17, wherein the first or the second insert includes a removable obturator for atraumatic insertion of the respective insert into the associated damaged ureter or urethra, the obturator having an inflatable balloon tip portion, a tubular central portion, and a bottom end portion, the bottom end portion including a valve for inflating or deflating the balloon tip portion.

24. An obturator for removing a stricture in an associated luminal structure, the obturator comprising:
a first body including a tip and a base, the tip including an orifice and the base including a port for receiving a first fluid, the orifice and the port being in fluid communication;
a first inflatable balloon disposed at the tip of the first body;
a second body including a tip and a base, the tip including an orifice and the base including a port for receiving a second fluid, the orifice and the port being in fluid communication;
a second inflatable balloon disposed at the tip of the second body; and wherein, the second balloon is disposed below the first balloon and the second body is slidably engaged with the first body.

25. The obturator of claim 24, wherein the first balloon includes a magnetically responsive member.

26. The obturator of claim 24, wherein the second balloon includes a magnetically responsive member.

27. The obturator of claim 24, wherein the second balloon includes a transverse diameter that is substantially that of the second body when the second balloon is in a deflated state.

28. The obturator of claim 24, wherein the base of the first and the second bodies include a valve for selectively permitting fluid flow from the port to the orifice of the respective first and second bodies.

29. A method for removing a stricture from an associated human luminal structure, the method comprising the steps of:
providing an insert including an insert body, a tip, a base and a bore extending through the insert body from the base to the tip;
providing an obturator including a first body having a first inflatable balloon and a second body having a second inflatable balloon, the second balloon being disposed below the first balloon and the first body being slidably engaged within the second body;
inserting the obturator into the bore of the insert with the first and second balloons in a deflated state;
inflating the second balloon;
introducing the insert and the obturator into a proximal portion of the associated luminal structure;
advancing the insert and the obturator until the tip of the insert is proximal to the associated stricture;
deflating the second balloon;
retracting the second body until the second balloon is disposed within the bore of the insert;
advancing the first balloon through the associated stricture;
inflating the first balloon;
retracting the first body and the first balloon and urging the associated stricture into a concave recess in the tip of the insert;
activating a suturing device within the insert and joining the proximal portion of the associated luminal structure adjacent the associated stricture to a distal portion of the associated luminal structure adjacent the associated stricture;
separating the associated stricture from the adjacent proximal and the distal portions of the associated luminal structure; and
withdrawing the insert and obturator from the associated luminal structure.

30. The method of claim 29, further including the steps of providing an electromagnet in the tip of the insert and providing a magnetically responsive member in the first inflatable balloon.

31. The method of claim 30, wherein the step of retracting the first body and first balloon further includes activating the electromagnet and electromagnetically urging the magnetically responsive member in the first inflatable balloon towards the concave recess of the tip of the insert.
